(19) United States
(12) Patent Application Publication
(10) Pub. No.: US 2026/0301141 A1
(43) Pub. Date: Oct. 1, 2026

(54) SYSTEM AND METHOD FOR AUTOMATING CORROSION DETECTION

(71) Applicant: Schlumberger Technology Corporation, Sugar Land, TX (US)

(72) Inventors: Fei Song, Houston, TX (US); Juan Alvarez, Houston, TX (US); Edward Gutierrez, JR., Houston, TX (US); Judy Zhuqing Dong, Houston, TX (US)

(21) Appl. No.: 19/095,356

(22) Filed: Mar. 31, 2025

(51) Int. Cl. G06T 7/00; G01N 33/20; G06N 3/0475; G06N 3/096; G06V 10/774; G06V 10/82

(52) U.S. Cl. CPC G06T 7/0002; G01N 33/20; G06N 3/0475; G06N 3/096; G06V 10/774; G06V 10/82; G06T 2207/20081; G06T 2207/20084

(57) ABSTRACT

A method for automating corrosion detection for process equipment includes receiving a raw image of the process equipment from an imaging device. The method also includes generating input prompts based upon the raw image, where the input prompts are generated by a prompt engine. The method further includes identifying, with a pretrained generative artificial intelligence (AI) model, one or more segmentations of corrosion on the process equipment based on the input prompts and the raw image. The method may also include determining a corrosion condition for each segmentation of the one or more segmentations of corrosion.

### BACKGROUND

Oil and gas process equipment includes a wide array of mechanical and structural components utilized throughout the exploration, production, processing, and transportation of hydrocarbons. These components commonly include valves, pumps, membrane separators, pressure vessels, storage tanks, compressors, heat exchangers, dehydration units, piping, flanges, fasteners, breakers, and the like. An operational concern for oil and gas process equipment—particularly those utilized in harsh, high-moisture, or chemically aggressive environments, such as offshore platforms, and high-salinity or high-sulfur production fields—is corrosion. Corrosion is an electrochemical process in which metals deteriorate due to interactions with their surroundings, especially in the presence of water, salts, carbon dioxide, hydrogen sulfide, and other corrosive agents. These conditions may accelerate metal degradation and compromise the structural integrity and safety of the oil and gas process equipment. Conventional methods for detecting corrosion are largely manual and rely on visual examination or other nondestructive evaluation (NDE) techniques. These approaches are often time-consuming, labor-intensive, and may yield inconsistent or incomplete results due to human subjectivity and limited coverage. As a result, damage may often go undetected, increasing the likelihood of unexpected equipment failures, unplanned shutdowns, and costly maintenance.

Accordingly, there is a need for improved methods and systems for automating corrosion detection in oil and gas process equipment.

### SUMMARY

A method for automating corrosion detection for process equipment is disclosed. The method includes receiving a raw image of the process equipment from an imaging device. The method also includes generating input prompts based upon the raw image. The input prompts are generated by a prompt engine. The method further includes identifying, with a pretrained generative artificial intelligence (AI) model, one or more segmentations of corrosion on the process equipment based on the input prompts and the raw image. The method also include determining a corrosion condition for each segmentation of the one or more segmentations of corrosion.

A computing system is also disclosed. The computing system includes one or more processors and a method system. The method system includes one or more non-transitory computer-readable media storing instructions that, when executed by at least one of the one or more processors, cause the computing system to perform operations. The operations include receiving a raw image of the process equipment from an imaging device. The operations also includes generating input prompts based upon the raw image. The input prompts are generated by a prompt engine. The operations further includes identifying, with a pretrained generative artificial intelligence (AI) model, one or more segmentations of corrosion on the process equipment based on the input prompts and the raw image. The operations also include determining a corrosion condition for each segmentation of the one or more segmentations of corrosion. The operations further include displaying an annotated image comprising the raw image and the one or more segmentations of corrosion.

A non-transitory computer-readable medium is also disclosed. The medium stores instructions that, when executed by one or more processors of a computing system, cause the computing system to perform operations. The operations include receiving a raw image of the process equipment from an imaging device. The operations also includes generating input prompts based upon the raw image. The input prompts are generated by a prompt engine. The operations further includes identifying, with a pretrained generative artificial intelligence (AI) model, one or more segmentations of corrosion on the process equipment based on the input prompts and the raw image. The operations also include determining a corrosion condition for each segmentation of the one or more segmentations of corrosion. The operations further include displaying an annotated image comprising the raw image and the one or more segmentations of corrosion. The operations also include performing an action in response to the respective corrosion condition of at least one segmentation of the one or more segmentations of corrosion.

It will be appreciated that this summary is intended merely to introduce some aspects of the present methods, systems, and media, which are more fully described and/or claimed below. Accordingly, this summary is not intended to be limiting.

### BRIEF DESCRIPTION OF THE DRAWINGS

The accompanying drawings, which are incorporated in and constitute a part of this specification, illustrate embodiments of the present teachings and together with the description, serve to explain the principles of the present teachings. In the figures:

FIG. 1 illustrates an example of a system that includes various management components to manage various aspects of a geologic environment, according to an embodiment.

FIG. 2 illustrates a schematic diagram of an exemplary system and an associated workflow of the system for automating corrosion detection for process equipment, according to an embodiment.

FIG. 3A illustrates an exemplary raw image of process equipment including background points and foreground points from a keypoint detector of a prompt engine, according to an embodiment.

FIG. 3B illustrates the raw image of FIG. 3A including the process equipment identified by the prompt engine, according to an embodiment.

FIG. 4A illustrates an exemplary raw image of process equipment including bounding boxes from an object detector of a prompt engine, according to an embodiment.

FIG. 4B illustrates the raw image of FIG. 4A including one or more segmentations of corrosion identified by a generative artificial intelligence model, according to an embodiment.

FIG. 5A illustrates an exemplary raw image of process equipment including masks from a segmentation model, according to an embodiment.

FIG. 5B illustrates the raw image of FIG. 5A including annotations that indicate a degree of corrosion for each of the one or more segmentations of corrosion, according to an embodiment.

FIG. 6A illustrates an exemplary raw image of process equipment, according to an embodiment.

FIG. 6B illustrates the raw image of FIG. 6A including text annotation indicating a number of segmentations of corrosion detected, according to an embodiment.

FIG. 7 illustrates an exemplary raw image of process equipment without corrosion, according to an embodiment.

FIG. 8 illustrates a flowchart of a method for automating corrosion detection for process equipment, according to an embodiment.

FIG. 9 illustrates a schematic view of a computing system for performing at least a portion of the method(s) described herein, according to an embodiment.

### DETAILED DESCRIPTION

Reference will now be made in detail to embodiments, examples of which are illustrated in the accompanying drawings and figures. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first object or step could be termed a second object or step, and, similarly, a second object or step could be termed a first object or step, without departing from the scope of the present disclosure. The first object or step, and the second object or step, are both, objects or steps, respectively, but they are not to be considered the same object or step.

The terminology used in the description herein is for the purpose of describing particular embodiments and is not intended to be limiting. As used in this description and the appended claims, the singular forms “a,” “an” and “the” are intended to include the plural forms as well, unless the context clearly indicates otherwise. It will also be understood that the term “and/or” as used herein refers to and encompasses any possible combinations of one or more of the associated listed items. It will be further understood that the terms “includes,” “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Further, as used herein, the term “if” may be construed to mean “when” or “upon” or “in response to determining” or “in response to detecting,” depending on the context.

Attention is now directed to processing procedures, methods, techniques, and workflows that are in accordance with some embodiments. Some operations in the processing procedures, methods, techniques, and workflows disclosed herein may be combined and/or the order of some operations may be changed.

### System Overview

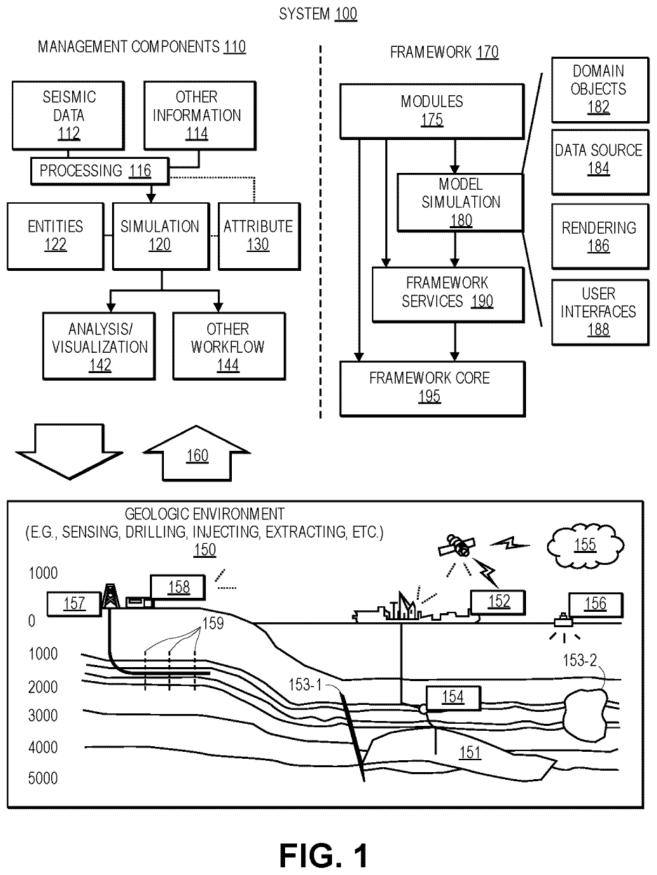

FIG. 1 illustrates an example of a system 100 that includes various management components 110 to manage various aspects of a geologic environment 150 (e.g., an environment that includes a sedimentary basin, a reservoir 151, one or more faults 153-1, one or more geobodies 153-2, etc.). For example, the management components 110 may allow for direct or indirect management of sensing, drilling, injecting, extracting, etc., with respect to the geologic environment 150. In turn, further information about the geologic environment 150 may become available as feedback 160 (e.g., optionally as input to one or more of the management components 110).

In the example of FIG. 1, the management components 110 include a seismic data component 112, an additional information component 114 (e.g., well/logging data), a processing component 116, a simulation component 120, an attribute component 130, an analysis/visualization component 142 and a workflow component 144. In operation, seismic data and other information provided per the components 112 and 114 may be input to the simulation component 120.

In an example embodiment, the simulation component 120 may rely on entities 122. Entities 122 may include earth entities or geological objects such as wells, surfaces, bodies, reservoirs, etc. In the system 100, the entities 122 can include virtual representations of actual physical entities that are reconstructed for purposes of simulation. The entities 122 may include entities based on data acquired via sensing, observation, etc. (e.g., the seismic data 112 and other information 114). An entity may be characterized by one or more properties (e.g., a geometrical pillar grid entity of an earth model may be characterized by a porosity property). Such properties may represent one or more measurements (e.g., acquired data), calculations, etc.

In an example embodiment, the simulation component 120 may operate in conjunction with a software framework such as an object-based framework. In such a framework, entities may include entities based on pre-defined classes to facilitate modeling and simulation. A commercially available example of an object-based framework is the MICROSOFT®. NET® framework (Redmond, Washington), which provides a set of extensible object classes. In the . NET® framework, an object class encapsulates a module of reusable code and associated data structures. Object classes can be used to instantiate object instances for use in by a program, script, etc. For example, borehole classes may define objects for representing boreholes based on well data.

In the example of FIG. 1, the simulation component 120 may process information to conform to one or more attributes specified by the attribute component 130, which may include a library of attributes. Such processing may occur prior to input to the simulation component 120 (e.g., consider the processing component 116). As an example, the simulation component 120 may perform operations on input information based on one or more attributes specified by the attribute component 130. In an example embodiment, the simulation component 120 may construct one or more models of the geologic environment 150, which may be relied on to simulate behavior of the geologic environment 150 (e.g., responsive to one or more acts, whether natural or artificial). In the example of FIG. 1, the analysis/visualization component 142 may allow for interaction with a model or model-based results (e.g., simulation results, etc.). As an example, output from the simulation component 120 may be input to one or more other workflows, as indicated by a workflow component 144.

As an example, the simulation component 120 may include one or more features of a simulator such as the ECLIPSE™ reservoir simulator (SLB, Houston Texas), the INTERSECT™ reservoir simulator (SLB, Houston Texas), etc. As an example, a simulation component, a simulator, etc. may include features to implement one or more meshless techniques (e.g., to solve one or more equations, etc.). As an example, a reservoir or reservoirs may be simulated with respect to one or more enhanced recovery techniques (e.g., consider a thermal process such as SAGD, etc.).

As an example, the simulation component 120 may include one or more features of a simulator such as SYMMETRY software (SLB, Houston, Texas). More particularly, SYMMETRY may process workflows in a single integrated environment with accurate thermodynamic fluid representation and consistent modeling across multiple disciplines including process, production, and HSE. The simulator integrates steady-state and transient (e.g., dynamic) analyses that can be tailored for each domain. This approach enables users to optimize processes in upstream, midstream, and downstream sectors while maximizing profits and minimizing capital expenditures. It may also help reduce emissions, energy consumption, and waste.

As an example, the simulation component 120 may include one or more features of a simulator such as PIPESIM (SLB, Houston, Texas). More particularly, PIPESIM is steady-state multiphase flow simulator that incorporates the three areas of flow modeling: multiphase flow, heat transfer and fluid behavior.

As an example, the simulation component 120 may include one or more features of a simulator such as OLGA™ (SLB, Houston, Texas). More particularly, OLGA™ is a dynamic multiphase flow simulator that models transient flow (e.g., time-dependent behaviors) to maximize production potential. Transient modeling is a component for feasibility studies and field development design. Dynamic simulation is useful in deep water and is used in both offshore and onshore developments to investigate transient behavior in pipelines and wellbores. Transient simulation with the OLGA™ simulator provides an added dimension to steady-state analysis by predicting system dynamics, such as time-varying changes in flow rates, fluid compositions, temperature, solids deposition, and operational changes.

In an example embodiment, the management components 110 may include features of a commercially available framework such as the PETREL® seismic to simulation software framework (SLB, Houston, Texas). The PETREL® framework provides components that allow for optimization of exploration and development operations. The PETREL® framework includes seismic to simulation software components that can output information for use in increasing reservoir performance, for example, by improving asset team productivity. Through use of such a framework, various professionals (e.g., geophysicists, geologists, and reservoir engineers) can develop collaborative workflows and integrate operations to streamline processes. Such a framework may be considered an application and may be considered a data-driven application (e.g., where data is input for purposes of modeling, simulating, etc.).

In an example embodiment, various aspects of the management components 110 may include add-ons or plug-ins that operate according to specifications of a framework environment. For example, a commercially available framework environment marketed as the OCEAN® framework environment (SLB, Houston, Texas) allows for integration of add-ons (or plug-ins) into a PETREL® framework workflow. The OCEAN® framework environment leverages .NET® tools (Microsoft Corporation, Redmond, Washington) and offers stable, user-friendly interfaces for efficient development. In an example embodiment, various components may be implemented as add-ons (or plug-ins) that conform to and operate according to specifications of a framework environment (e.g., according to application programming interface (API) specifications, etc.).

FIG. 1 also shows an example of a framework 170 that includes a model simulation layer 180 along with a framework services layer 190, a framework core layer 195 and a modules layer 175. The framework 170 may include the commercially available OCEAN® framework where the model simulation layer 180 is the commercially available PETREL® model-centric software package that hosts OCEAN® framework applications. In an example embodiment, the PETREL® software may be considered a data-driven application. The PETREL® software can include a framework for model building and visualization.

As an example, a framework may include features for implementing one or more mesh generation techniques. For example, a framework may include an input component for receipt of information from interpretation of seismic data, one or more attributes based at least in part on seismic data, log data, image data, etc. Such a framework may include a mesh generation component that processes input information, optionally in conjunction with other information, to generate a mesh.

In the example of FIG. 1, the model simulation layer 180 may provide domain objects 182, act as a data source 184, provide for rendering 186 and provide for various user interfaces 188. Rendering 186 may provide a graphical environment in which applications can display their data while the user interfaces 188 may provide a common look and feel for application user interface components.

As an example, the domain objects 182 can include entity objects, property objects and optionally other objects. Entity objects may be used to geometrically represent wells, surfaces, bodies, reservoirs, etc., while property objects may be used to provide property values as well as data versions and display parameters. For example, an entity object may represent a well where a property object provides log information as well as version information and display information (e.g., to display the well as part of a model).

In the example of FIG. 1, data may be stored in one or more data sources (or data stores, generally physical data storage devices), which may be at the same or different physical sites and accessible via one or more networks. The model simulation layer 180 may be configured to model projects. As such, a particular project may be stored where stored project information may include inputs, models, results and cases. Thus, upon completion of a modeling session, a user may store a project. At a later time, the project can be accessed and restored using the model simulation layer 180, which can recreate instances of the relevant domain objects.

In the example of FIG. 1, the geologic environment 150 may include layers (e.g., stratification) that include a reservoir 151 and one or more other features such as the fault 153-1, the geobody 153-2, etc. As an example, the geologic environment 150 may be outfitted with any of a variety of sensors, detectors, actuators, etc. For example, equipment 152 may include communication circuitry to receive and to transmit information with respect to one or more networks 155. Such information may include information associated with downhole equipment 154, which may be equipment to acquire information, to assist with resource recovery, etc. Other equipment 156 may be located remote from a well site and include sensing, detecting, emitting or other circuitry. Such equipment may include storage and communication circuitry to store and to communicate data, instructions, etc. As an example, one or more satellites may be provided for purposes of communications, data acquisition, etc. For example, FIG. 1 shows a satellite in communication with the network 155 that may be configured for communications, noting that the satellite may additionally or instead include circuitry for imagery (e.g., spatial, spectral, temporal, radiometric, etc.).

FIG. 1 also shows the geologic environment 150 as optionally including equipment 157 and 158 associated with a well that includes a substantially horizontal portion that may intersect with one or more fractures 159. For example, consider a well in a shale formation that may include natural fractures, artificial fractures (e.g., hydraulic fractures) or a combination of natural and artificial fractures. As an example, a well may be drilled for a reservoir that is laterally extensive. In such an example, lateral variations in properties, stresses, etc. may exist where an assessment of such variations may assist with planning, operations, etc. to develop a laterally extensive reservoir (e.g., via fracturing, injecting, extracting, etc.). As an example, the equipment 157 and/or 158 may include components, a system, systems, etc. for fracturing, seismic sensing, analysis of seismic data, assessment of one or more fractures, etc.

As mentioned, the system 100 may be used to perform one or more workflows. A workflow may be a process that includes a number of worksteps. A workstep may operate on data, for example, to create new data, to update existing data, etc. As an example, a may operate on one or more inputs and create one or more results, for example, based on one or more algorithms. As an example, a system may include a workflow editor for creation, editing, executing, etc. of a workflow. In such an example, the workflow editor may provide for selection of one or more pre-defined worksteps, one or more customized worksteps, etc. As an example, a workflow may be a workflow implementable in the PETREL® software, for example, that operates on seismic data, seismic attribute(s), etc. As an example, a workflow may be a process implementable in the OCEAN® framework. As an example, a workflow may include one or more worksteps that access a module such as a plug-in (e.g., external executable code, etc.).

The present disclosure includes a system and method for automating corrosion detection for process equipment in oil and gas production facilities. Particularly, the present disclosure is directed to a system and method for automating corrosion detection in oil and gas process equipment. Illustrative oil and gas process equipment may be or include, but is not limited to, a valve, a pump, a membrane separator, a tank, a vessel, a compressor, a heat exchanger, a dehydration unit, a flange, a fastener, a breaker, or the like, or any combination thereof. While the present disclosure makes references to oil and gas process equipment, it should be appreciated that the system and method disclosed herein may be equally applicable to other process equipment unrelated to oil and gas.

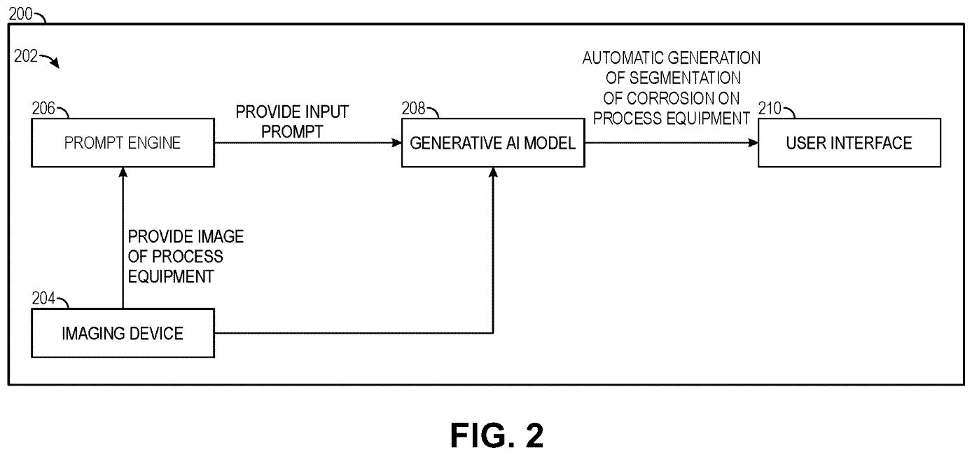

FIG. 2 illustrates a schematic diagram of an exemplary system 200 and an associated workflow 202 of the system 200 for automating corrosion detection for process equipment and/or components thereof, according one or more embodiments. The system 200 and the associated workflow 202 may generally be capable of or configured to capture an image (e.g., a raw image) of the process equipment, identify segmentations of corrosion on the process equipment, and determine a corrosion condition for each of the segmentations of corrosion. The system 200 may include one or more of an imaging device 204, a prompt engine 206, a generative artificial intelligence (AI) model 208, a user interface 210, or any combination thereof. For example, as illustrated in FIG. 2, the system 200 may include the imaging device 204 operably coupled with the prompt engine 206 and/or the generative AI model 208, the prompt engine 206 operably coupled with the generative AI model 208, and the generative AI model 208 operably coupled with the user interface 210. As further described herein, the workflow 202 of the system 200 may generally include capturing an image (e.g., raw image which pixel may be defined by the amount of red, green, and blue (RGB) colors, or by other color models) of the process equipment with the imaging device 204 and directing the image to the prompt engine 206. The workflow 202 may also include generating input prompts based upon the image, where the input prompts are generated by the prompt engine 206, and directing or outputting the input prompts to the generative AI model 208. The workflow 202 may further include automatically identifying one or more segmentations of corrosion on the process equipment with the generative AI model 208, and displaying the segmentations of corrosion via the user interface 210.

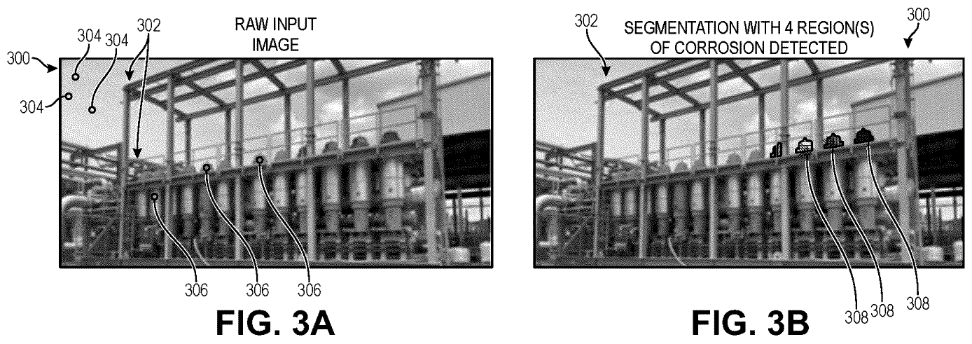

The imaging device 204 may be any suitable device capable of or configured to capture the image, such as a raw image, of the process equipment. For example, the imaging device 204 may be or include one or more cameras capable of or configured to capture a raw image of the process equipment. As used herein, the expression “raw image” may refer to an original, high-resolution, uncompressed file or image, or a pixel array prior to normalization, augmentation, or preprocessing. FIGS. 3A, 4A, 5A, 6A, and 7 illustrate exemplary raw images 300, 400, 500, 600, 700 of the process equipment captured or provided by the imaging device 204. In at least one embodiment, the imaging device 204 may be or include an autonomous or semi-autonomous imaging device including the one or more cameras. The autonomous or semi-autonomous imaging device may be capable of or configured to autonomously or semi-autonomously capture images of the process equipment. As used herein, the expression “autonomous imaging device” or the like may refer to an imaging device that may operate independently without human intervention once initiated. As used herein, the expression “semi-autonomous imaging device” or the like may refer to an imaging device that may operate partially independently. For example, the semi-autonomous imaging device may perform some functions automatically and some functions with human intervention (e.g., control, oversight, etc.). Illustrative autonomous or semi-autonomous devices may be or include, but are not limited to, a robot, a drone, an agent, or the like, or any combination thereof. As illustrated in FIG. 2, the imaging device 204 may be operably coupled with the prompt engine 206 and/or the generative AI model 208 and capable of or configured to provide the images 300, 400, 500, 600, 700 of the process equipment to the prompt engine 206 and/or the generative AI model 208.

The prompt engine 206 may be capable of or configured to receive the images 300, 400, 500, 600, 700 of the process equipment from the imaging device 204, detect one or more objects in the images 300, 400, 500, 600, 700, and generate input prompts with the one or more objects detected. Illustrative objects that may be detected by the prompt engine 206 may be or include, but are not limited to, unknown objects, the process equipment, components of the process equipment, or the like, or any combination thereof. In at least one embodiment, the prompt engine 206 may include one or more detectors capable of or configured to receive the images 300, 400, 500, 600, 700 and detect and/or identify the respective objects (e.g., the process equipment or components thereof) in the images 300, 400, 500, 600, 700. The prompt engine 206 may utilize the objects detected by the one or more detectors to generate the input prompts. Illustrative input prompts may be or include, but are not limited to, one or more of point prompts, box prompts, mask prompts, text prompts, or a combination thereof. The one or more detectors of the prompt engine 206 may be or include, but are not limited to, a traditional computer vision-based detector, a machine learning-based detector, or the like, or any combination thereof. The traditional computer vision-based detector may include a scale-invariant feature transform (SIFT) model, template matching, or the like, or any combination thereof. The machine learning-based detector may be or include a machine learning-based keypoint detector (i.e., a keypoint detector), a machine learning-based object detector (i.e., an object detector), a machine learning-based segmentation model (i.e., a segmentation model), or the like, or any combination thereof.

In at least one embodiment, the machine learning-based detector of the prompt engine 206, including the keypoint detector, the object detector, and the segmentation model, may be obtained by training each of the detectors from scratch. In another embodiment, the machine learning-based detector may be obtained by using a pretrained backbone or pretrained detector. For example, the machine learning-based detector may be obtained by transfer learning or fine tuning a deep neural network of the pretrained backbone with a custom image dataset of the process equipment (e.g., the oil and gas process equipment).

The machine learning-based keypoint detector or keypoint detector of the prompt engine 206 may be capable of or configured to detect and/or identify the process equipment in the images 300, 400, 500, 600, 700 with background points, foreground points, or a combination thereof. For example, as illustrated in FIG. 3A, the keypoint detector may detect and/or identify one or more background points 304 and one or more foreground points 306. The background points 302 and/or the foreground points 304 from the keypoint detector may then be directed to or utilized by the prompt engine 206 to identify the process equipment and/or segmentations thereof in the image 300, as illustrated in FIG. 3B, and generate the prompt points 308 for the generative AI model 208 downstream of the prompt engine 206. Illustrative machine learning-based keypoint detectors may be or include, but are not limited to, CenterNet, You Only Look Once (YOLO), or the like, or any combination thereof.

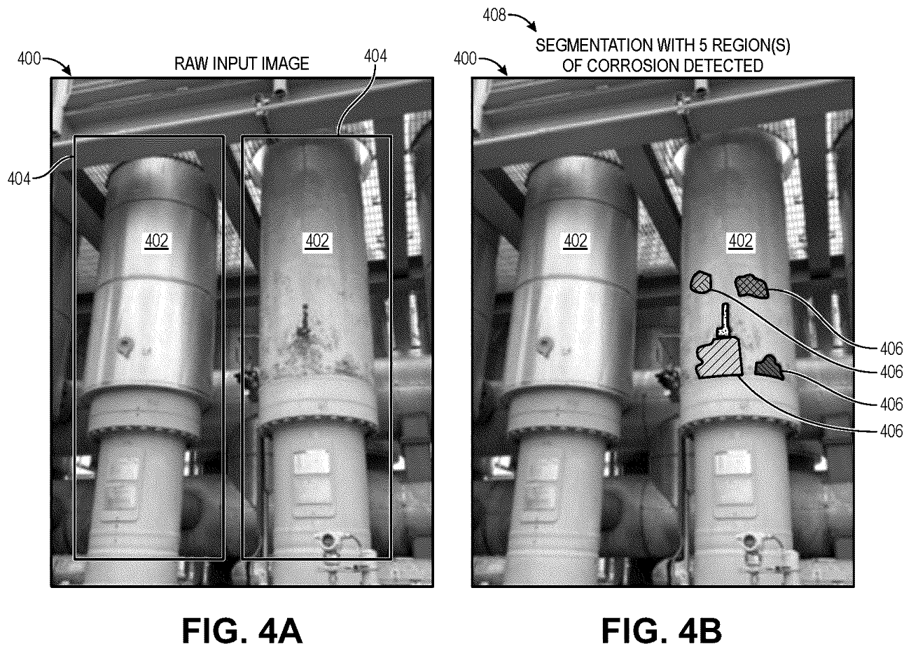

The machine learning-based object detector or object detector of the prompt engine 206 may be capable of or configured to detect and/or identify the process equipment in the images 300, 400, 500, 600, 700, and generate or construct one or more bounding boxes or spatial coordinates of the process equipment in or on the images 300, 400, 500, 600, 700. For example, as illustrated in FIG. 4A, the object detector may detect and/or identify piping 402 in the image 400, and construct the bounding boxes 404 of or about the piping 402 on the image 400. The bounding boxes 404 identifying the piping 402 may be output from the object detector and directed to or utilized by the prompt engine 206 to generate the box prompts for the generative AI model 208 downstream of the prompt engine 206. Illustrative object detectors may be or include, but are not limited to, Regions with Convolutional Neural Network (R-CNN), Fast R-CNN, Faster R-CNN, Single Shot MultiBox Detector, YOLO, RetinaNet, EfficientDet, CenterNet, or the like, or any combination thereof.

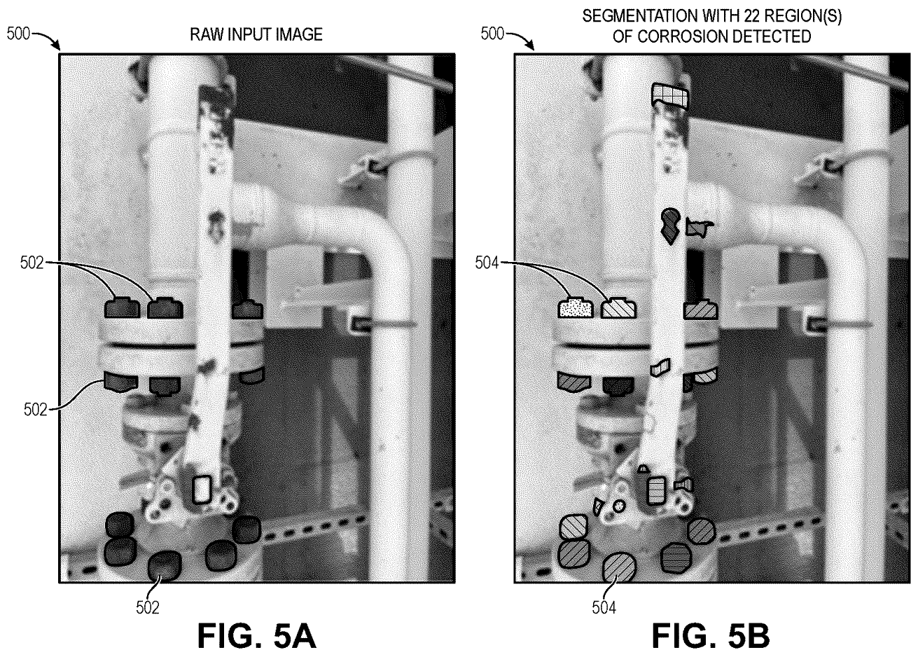

The machine learning-based segmentation model or segmentation model of the prompt engine 206 may be capable of or configured to detect and/or identify the process equipment in the images 300, 400, 500, 600, 700, and generate or construct one or more masks of the process equipment in or on the images 300, 400, 500, 600, 700. The masks may outline or substantially outline one or more of the objects in the images 300, 400, 500, 600, 700. For example, as illustrated in FIG. 5A, the segmentation model may detect and/or identify one or more nuts 502 in the image 500, and construct one or more of the masks 504 of the nuts 502 in or on the image 500. The masks 504 identifying the nuts 502 may be output from the segmentation model and directed to or utilized by the prompt engine 206 to generate the mask prompts for the generative AI model 208 downstream of the prompt engine 206. Illustrative segmentation models may be or include, but are not limited to Mask R-CNN, fully convolution network (FCN), YOLACT/YOLACT++, BlendMask, CenterMask, Cascade Mask R-CNN, or the like, or any combination thereof.

In at least one embodiment, the generative AI model 208 may receive the images 300, 400, 500, 600, 700 from the prompt engine 206, the imaging device 204, or a combination thereof. For example, as illustrated in FIG. 2, the generative AI model 208 may be operably coupled with the prompt engine 206 and the imaging device 204 and configured to receive the images 300, 400, 500, 600, 700 therefrom. The generative AI model 208 may be capable of or configured to autonomously identify an unknown object in the respective images 300, 400, 500, 600, 700, and apply a zero-shot generalization model to the unknown object. The generative AI model 208 may also be capable of or configured to receive the images 300, 400, 500, 600, 700 of the process equipment and the input prompts generated by the prompt engine 206, and autonomously identify one or more segmentations of corrosion on the process equipment based on the input prompts and the images 300, 400, 500, 600, 700. For example, the generative AI model 208 may be capable of or configured to receive one or more of the prompt points, the box prompts, the mask prompts, the text prompts, or any combination thereof generated by the prompt engine 206, and autonomously identify the one or more segmentations of corrosion on the process equipment based on the input prompts and the images 300, 400, 500, 600, 700. As illustrated in FIG. 4B, the generative AI model 208 may be capable of or configured to receive the box prompts generated by the prompt engine 206 from the bounding boxes 404 and the image 400 of the piping 402, and identify the one or more segmentations of corrosion 406 on the piping 402 based on the box prompts and the image 400. Examples of the generative AI model may be or include, but are not limited to, zero-shot vision models for generating promptable image masks, such as Segment Anything Model or Segment Anything Model 2, CLIPSeg, YOLOE, or the like, or any combination thereof.

The generative AI model 208 may be a pretrained generative AI model. In at least one embodiment, the generative AI model 208 may not be further trained or fine-tuned. For example, the generative AI model 208 may be the pretrained generative AI model utilized “off-the-shelf” without additional training, customization, and/or fine tuning. In another embodiment, the generative AI model 208 may be further trained and/or fine-tuned. For example, the pretrained generative AI model may be fine-tuned by extracting one or more key components of the pretrained generative AI model, creating training data including a custom image dataset of the process equipment, namely, the oil and gas process equipment, setting up an optimizer and a scheduler for the pretrained generative AI model, and iteratively updating the pretrained generative AI model with the training data including the custom image dataset. The one or more key components extracted from the generative AI model 208 may be or include, but are not limited to, one or more of an image encoder, a prompt encoder, a mask decoder, or any combination thereof.

The generative AI model 208 may be capable of or configured to autonomously determine a corrosion condition for each of the one or more segmentations of corrosion 406. Illustrative corrosion conditions may be or include, but are not limited to, a degree of corrosion, a corrosion pattern, a type of corrosion, or the like, or any combination thereof. Illustrative degrees of corrosion may be or include, but is not limited to, no corrosion, minor corrosion, moderate corrosion, severe corrosion, or the like. Illustrative types of corrosion may be or include, but are not limited to, general or uniform corrosion (e.g., rusting), localized corrosion (e.g., pitting, crevice, intergranular, etc.), stress corrosion cracking, or the like, or any combination thereof. The generative AI model 208 may be capable of or configured to output the one or more segmentations of corrosion 406 and/or the corrosion condition of each of the segmentations of corrosion, and direct the output to the user interface 210.

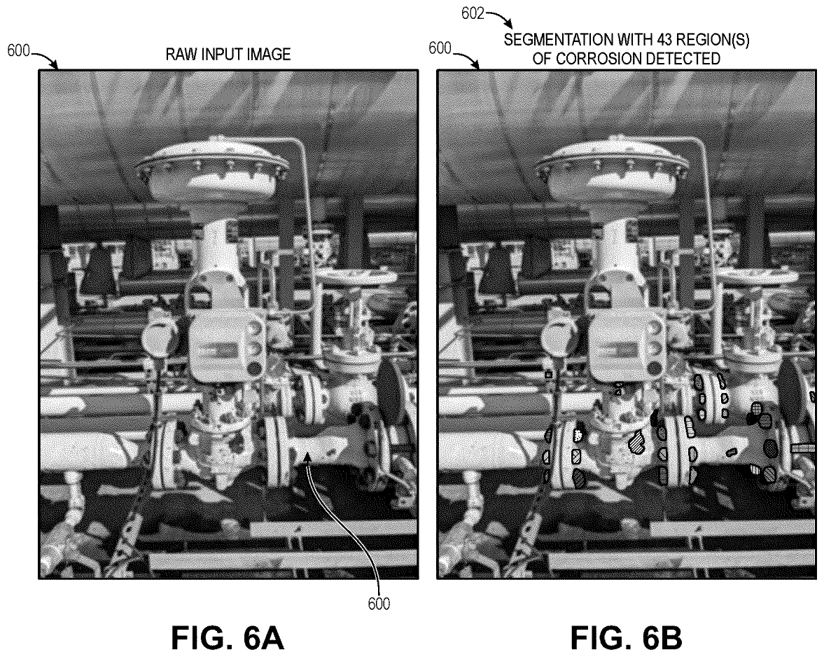

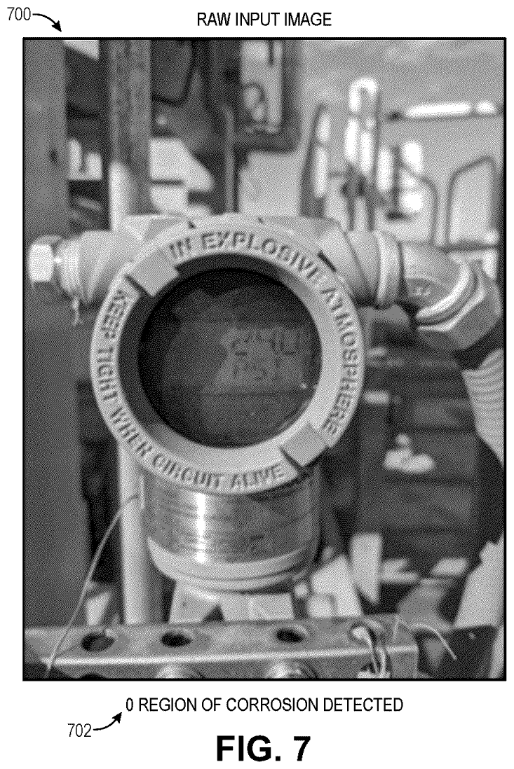

The user interface 210 may be capable of or configured to allow a user, such as a well engineer, to view the segmentations of corrosion 406, the corrosion condition of each of the segmentations of corrosion, or a combination thereof. For example, the user interface 210 may be or include a display capable of or configured to allow the user to view the segmentations of corrosion 406 and the respective corrosion conditions thereof. In at least one embodiment, the user interface 210 may display an annotated image including the respective image 300, 400, 500, 600, 700 of the process equipment and the segmentations of corrosion 406. For example, as illustrated in FIG. 4B, the user interface 210 may display the image 400 of the piping 402 including annotations of the segmentations of corrosion 406. The segmentations of corrosion 406 may be displayed as an overlay on the image 400. The user interface 210 may display a particular annotation on the image 400 that encodes each of the corrosion conditions with a unique or distinguishing visual feature. For example, each of the degrees of corrosion may be represented by a distinguishing color, hatching, text, or the like, or any combination thereof. For example, as illustrated in FIG. 5B, a minor degree of corrosion may be indicated with a first type of hatching and a severe degree of corrosion may be indicated with a second type of hatching. The user interface 210 may also annotate the image 400 with a count or number of segmentations of corrosion. For example, as illustrated in FIG. 4B, the user interface 210 may annotate the image 400 with text or a text box 408 indicating the number of segmentations of corrosion. In another example, illustrated in FIG. 6B, the user interface 210 may annotate the image 600 with text 602 indicating the number of segmentations of corrosion. The user interface 210 may also annotate the image with an indication of whether corrosion is present or absent. For example, as illustrated in FIG. 7, the user interface 210 may annotate the image 700 with text 702 indicating the absence of corrosion.

### Exemplary Method

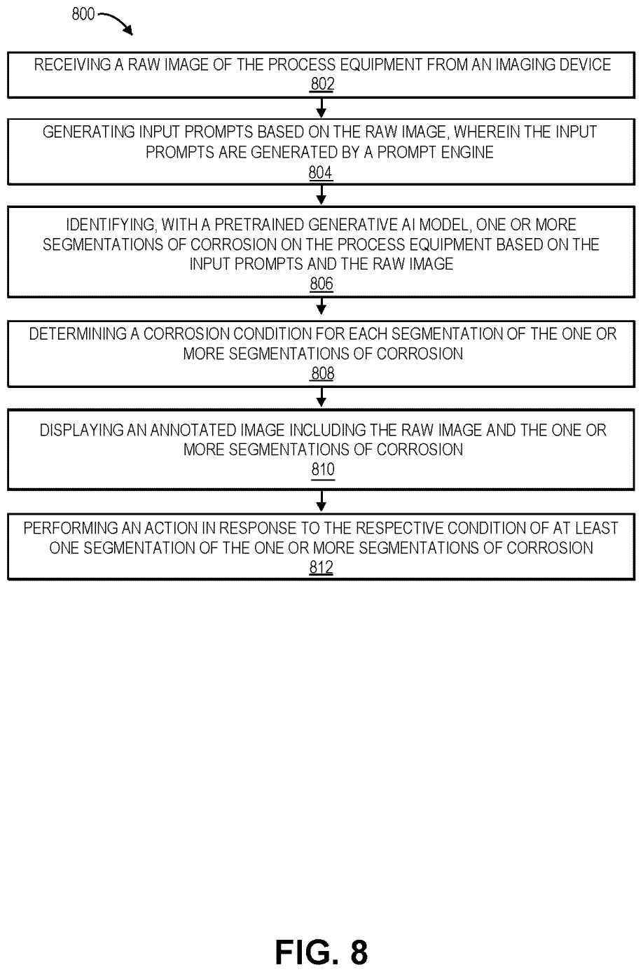

FIG. 8 illustrates a flowchart of a method 800 for automating corrosion detection for process equipment, according to an embodiment. An illustrative order of the method 800 is provided below; however, one or more portions of the method 800 may be performed in a different order, simultaneously, repeated, or omitted. At least a portion of the method 800 may be performed using a computing system.

The method 800 may include receiving a raw image of the process equipment from an imaging device, as at 802. The process equipment may be or include oil and gas process equipment. The process equipment may include one or more of a valve, a pump, a membrane separator, a tank, a vessel, a compressor, a heat exchanger, a dehydration unit, a flange, a fastener, a breaker, or any combination thereof. The imaging device may include a camera. The imaging device may be or include an autonomous device. The autonomous device may include an autonomous robot or an autonomous agent.

The method 800 may also include generating input prompts based upon the raw image, where the input prompts are generated by a prompt engine, as at 804. The input prompts may be or include one or more of point prompts, box prompts, mask prompts, text prompts, or any combination thereof. Generating the input prompts with the prompt engine and the raw image may include identifying one or more objects in the raw image with a detector of the prompt engine, and generating the input prompts with the prompt engine based on the one or more objects identified by the detector. The detector may be a machine learning-based detector or a traditional vision-based detector. The machine learning-based detector may include one or more of a keypoint detector, an object detector, a segmentation model, or any combination thereof. The keypoint detector may be or include, but is not limited to, CenterNet, You Only Look Once (YOLO), or the like, or any combination thereof. The object detector may be or include, but is not limited to, Regions with Convolutional Neural Network (R-CNN), Fast R-CNN, Faster R-CNN, Single Shot MultiBox Detector, YOLO, RetinaNet, EfficientDet, CenterNet, or the like, or any combination thereof. The segmentation model may be or include, but is not limited to, Mask R-CNN, fully convolution network (FCN), YOLACT/YOLACT++, BlendMask, CenterMask, Cascade Mask R-CNN, or the like, or any combination thereof. The traditional computer vision-based detector may include a scale-invariant feature transform (SIFT) model, template matching, or the like, or any combination thereof. Generating the input prompts with the prompt engine may include fine tuning a deep neural network of the machine learning-based detector with a custom image dataset. The custom image dataset may include images of the process equipment, such as oil and gas process equipment.

The method 800 may also include identifying, with a pretrained generative artificial intelligence (AI) model, one or more segmentations of corrosion on the process equipment based on the input prompts and the raw image, as at 806. Identifying the one or more segmentations of corrosion on the process equipment may include identifying an unknown object in the raw image, and applying a zero-shot generalization model to the unknown object. In at least one example, identifying the one or more segmentations of corrosion on the process equipment may exclude fine tuning the pretrained generative AI model. In another example, identifying the one or more segmentations of corrosion on the process equipment may also include fine tuning the pretrained generative AI model. Fine tuning the pretrained generative AI model may include extracting one or more key components of the pretrained generative AI model. The one or more key components of the pretrained generative AI model may include one or more of an image encoder, a prompt encoder, a mask decoder, or the like, or any combination thereof. Fine tuning the pretrained generative AI model may also include creating a custom image dataset for the pretrained generative AI model. The custom image dataset may include images of oil and gas process equipment. Fine tuning the pretrained generative AI model may further include setting up an optimizer and a schedule for the pretrained generative AI model. Fine tuning the pretrained generative AI model may also include iteratively updating the pretrained generative AI model with the custom image dataset.

The method 800 may also include determining a corrosion condition for each segmentation of the one or more segmentations of corrosion, as at 808. Determining the corrosion condition may include determining, for each segmentation of the one or more segmentations of corrosion, a degree of corrosion, a corrosion pattern, a type of corrosion, or any combination thereof. The degree of corrosion may be or include, but is not limited to, no corrosion, minor corrosion, moderate corrosion, or severe corrosion. The type of corrosion may be or include, but is not limited to, uniform corrosion, localized corrosion, stress corrosion cracking, or the like, or any combination thereof.

The method 800 may also include displaying an annotated image including the raw image and the one or more segmentations of corrosion, as at 810. The annotated image may indicate the respective corrosion condition of each segmentation of the one or more segmentations of corrosion. The annotated image may identify the corrosion condition of each segmentation of the one or more segmentations of corrosion. For example, the annotated image may identify a first corrosion condition with a first color and a second corrosion condition with a second color. More particularly, the annotated image may identify minor corrosion with the first color and sever corrosion with the second color. The annotated image may indicate a count of the segmentations of corrosion. The annotated image may also indicate the presence or absence of corrosion. The segmentations of corrosion may be displayed as an overlay on the raw image.

The method 800 may also include performing an action in response to the respective corrosion condition of at least one segmentation of the one or more segmentations of corrosion, as at 812. The action may include one or more remediation actions.

### Exemplary Computing System

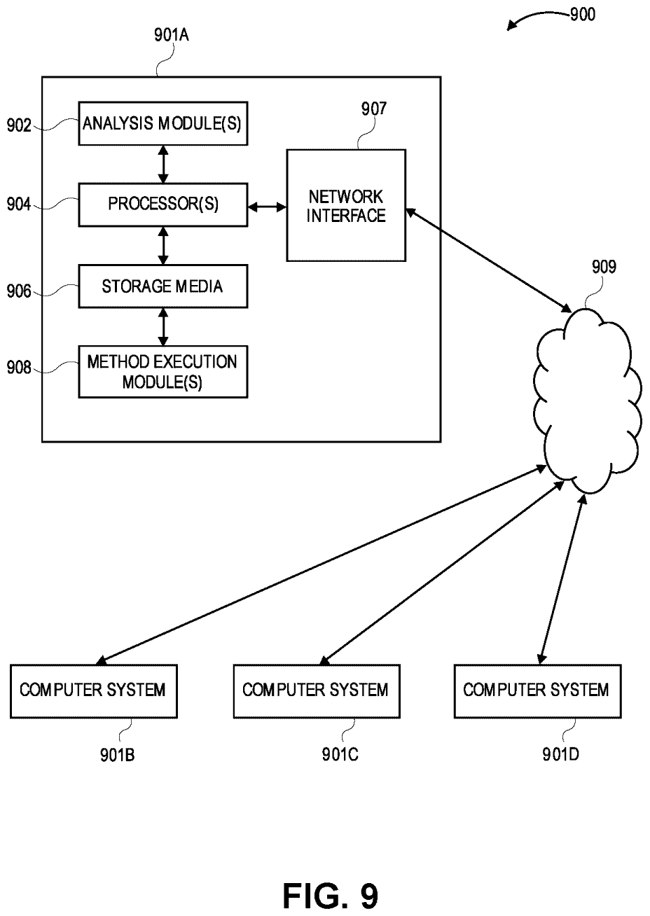

In some embodiments, the methods of the present disclosure may be executed by a computing system. FIG. 9 illustrates an example of such a computing system 900, in accordance with some embodiments. The computing system 900 may include a computer or computer system 901A, which may be an individual computer system 901A or an arrangement of distributed computer systems. The computer system 901A includes one or more analysis modules 902 that are configured to perform various tasks according to some embodiments, such as one or more methods disclosed herein. To perform these various tasks, the analysis module 902 executes independently, or in coordination with, one or more processors 904, which is (or are) connected to one or more storage media 906. The processor(s) 904 is (or are) also connected to a network interface 907 to allow the computer system 901A to communicate over a data network 909 with one or more additional computer systems and/or computing systems, such as 901B, 901C, and/or 901D (note that computer systems 901B, 901C and/or 901D may or may not share the same architecture as computer system 901A, and may be located in different physical locations, e.g., computer systems 901A and 901B may be located in a processing facility, while in communication with one or more computer systems such as 901C and/or 901D that are located in one or more data centers, and/or located in varying countries on different continents).

A processor may include a microprocessor, microcontroller, processor module or subsystem, programmable integrated circuit, programmable gate array, or another control or computing device.

The storage media 906 may be implemented as one or more computer-readable or machine-readable storage media. Note that while in the example embodiment of FIG. 9 storage media 906 is depicted as within computer system 901A, in some embodiments, storage media 906 may be distributed within and/or across multiple internal and/or external enclosures of computing system 901A and/or additional computing systems. Storage media 906 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLURAY® disks, or other types of optical storage, or other types of storage devices. Note that the instructions discussed above may be provided on one computer-readable or machine-readable storage medium, or may be provided on multiple computer-readable or machine-readable storage media distributed in a large system having possibly plural nodes. Such computer-readable or machine-readable storage medium or media is (are) considered to be part of an article (or article of manufacture). An article or article of manufacture may refer to any manufactured single component or multiple components. The storage medium or media may be located either in the machine running the machine-readable instructions, or located at a remote site from which machine-readable instructions may be downloaded over a network for execution.

In some embodiments, computing system 900 contains one or more method execution module(s) 908. In the example of computing system 900, computer system 901A includes the method execution module 908. In some embodiments, a single method execution module may be used to perform some aspects of one or more embodiments of the methods disclosed herein. In other embodiments, a plurality of method execution modules may be used to perform some aspects of methods herein.

It should be appreciated that computing system 900 is merely one example of a computing system, and that computing system 900 may have more or fewer components than shown, may combine additional components not depicted in the example embodiment of FIG. 9, and/or computing system 900 may have a different configuration or arrangement of the components depicted in FIG. 9. The various components shown in FIG. 9 may be implemented in hardware, software, or a combination of both hardware and software, including one or more signal processing and/or application specific integrated circuits.

Further, the steps in the processing methods described herein may be implemented by running one or more functional modules in information processing apparatus such as general purpose processors or application specific chips, such as ASICs, FPGAs, PLDs, or other appropriate devices. These modules, combinations of these modules, and/or their combination with general hardware are included within the scope of the present disclosure.

Computational interpretations, models, and/or other interpretation aids may be refined in an iterative fashion; this concept is applicable to the methods discussed herein. This may include use of feedback loops executed on an algorithmic basis, such as at a computing device (e.g., computing system 900, FIG. 9), and/or through manual control by a user who may make determinations regarding whether a given step, action, template, model, or set of curves has become sufficiently accurate for the evaluation of the subsurface three-dimensional geologic formation under consideration.

The foregoing description, for purpose of explanation, has been described with reference to specific embodiments. However, the illustrative discussions above are not intended to be exhaustive or limiting to the precise forms disclosed. Many modifications and variations are possible in view of the above teachings. Moreover, the order in which the elements of the methods described herein are illustrated and described may be re-arranged, and/or two or more elements may occur simultaneously. The embodiments were chosen and described in order to best explain the principles of the disclosure and its practical applications, to thereby enable others skilled in the art to best utilize the disclosed embodiments and various embodiments with various modifications as are suited to the particular use contemplated.

What is claimed is:

1. A method for automating corrosion detection for process equipment, the method comprising:
receiving a raw image of the process equipment from an imaging device;
generating input prompts based upon the raw image, wherein the input prompts are generated by a prompt engine;
identifying, with a pretrained generative artificial intelligence (AI) model, one or more segmentations of corrosion on the process equipment based on the input prompts and the raw image; and
determining a corrosion condition for each segmentation of the one or more segmentations of corrosion.

2. The method of claim 1, wherein generating the input prompts with the raw image and the prompt engine comprises:
identifying one or more objects in the raw image with a machine learning-based detector of the prompt engine; and
generating the input prompts with the prompt engine based on the one or more objects identified by the machine learning-based detector.

3. The method of claim 2, further comprising fine tuning a deep neural network of the machine learning-based detector of the prompt engine.

4. The method of claim 1, wherein the input prompts comprise one or more of point prompts, box prompts, mask prompts, text prompts, or a combination thereof.

5. The method of claim 1, wherein identifying the one or more segmentations of corrosion on the process equipment comprises fine tuning the pretrained generative AI model with a custom image dataset.

6. The method of claim 1, wherein identifying the one or more segmentations of corrosion does not comprise fine tuning the pretrained generative AI model.

7. The method of claim 1, wherein determining the corrosion condition for each segmentation of the one or more segmentations of corrosion comprises determining, for each segmentation of the one or more segmentations of corrosion, a degree of corrosion, a corrosion pattern, a type of corrosion, or any combination thereof.

8. The method of claim 1, further comprising displaying an annotated image comprising the raw image and the one or more segmentations of corrosion, wherein displaying the annotated image comprises displaying the one or more segmentations of corrosion as an overlay on the raw image.

9. The method of claim 8, wherein displaying the annotated image comprises displaying a count of the segmentations of corrosion on the annotated image.

10. The method of claim 1, wherein the imaging device is an autonomous device.

11. A computing system, comprising:
one or more processors; and
a memory system comprising one or more non-transitory computer-readable media storing instructions that, when executed by at least one of the one or more processors, cause the computing system to perform operations, the operations comprising:
receiving a raw image of process equipment from an imaging device;
generating input prompts based upon the raw image, wherein the input prompts are generated by a prompt engine;
identifying, with a pretrained generative artificial intelligence (AI) model, one or more segmentations of corrosion on the process equipment based on the input prompts and the raw image;
determining a corrosion condition for each segmentation of the one or more segmentations of corrosion; and
displaying an annotated image comprising the raw image and the one or more segmentations of corrosion.

12. The computing system of claim 11, wherein generating the input prompts with the raw image and the prompt engine comprises:
identifying one or more objects in the raw image with a detector of the prompt engine, wherein the detector is a machine learning-based detector;
generating the input prompts with the prompt engine based on the one or more objects identified by the detector, wherein the input prompts comprise one or more of point prompts, box prompts, mask prompts, text prompts, or a combination thereof; and
fine tuning a deep neural network of the machine learning-based detector with a custom dataset comprising images of oil and gas process equipment.

13. The computing system of claim 11, wherein identifying the one or more segmentations of corrosion on the process equipment comprises fine tuning the pretrained generative AI model, wherein fine tuning the pretrained generative AI model comprises:
extracting one or more key components of the pretrained generative AI model;
creating a custom image dataset for the pretrained generative AI model;
setting up an optimizer and a scheduler for the pretrained generative AI model; and
iteratively updating the pretrained generative AI model with the custom image dataset.

14. The computing system of claim 11, wherein identifying the one or more segmentations of corrosion does not comprise fine tuning the pretrained generative AI model.

15. The computing system of claim 11, wherein:
determining the corrosion condition for each segmentation of the one or more segmentations of corrosion comprises determining, for each segmentation of the one or more segmentations of corrosion, a degree of corrosion, a corrosion pattern, a type of corrosion, or any combination thereof; and
displaying the annotated image comprises:
displaying a count of the segmentations of corrosion on the annotated image;
displaying the respective corrosion condition of each segmentation of the one or more segmentations of corrosion on the annotated image; and
displaying the one or more segmentations of corrosion as an overlay on the raw image.

16. A non-transitory computer-readable medium storing instructions that, when executed by one or more processors of a computing system, cause the computing system to perform operations, the operations comprising:
receiving a raw image of process equipment from an imaging device;
generating input prompts based upon the raw image, wherein the input prompts are generated by a prompt engine;
identifying, with a pretrained generative artificial intelligence (AI) model, one or more segmentations of corrosion on the process equipment based on the input prompts and the raw image;
determining a corrosion condition for each segmentation of the one or more segmentations of corrosion;
displaying an annotated image comprising the raw image and the one or more segmentations of corrosion; and
performing an action in response to the respective corrosion condition of at least one segmentation of the one or more segmentations of corrosion.

17. The non-transitory computer-readable medium of claim 16, wherein the process equipment comprises oil and gas process equipment, wherein the oil and gas process equipment comprises one or more of a valve, a pump, a membrane separator, a tank, a vessel, a compressor, a heat exchanger, a dehydration unit, a flange, a fastener, a breaker, or any combination thereof, and wherein the imaging device is an autonomous device comprising a camera.

18. The non-transitory computer-readable medium of claim 16, wherein generating the input prompts with the raw image and the prompt engine comprises:
identifying one or more objects in the raw image with a detector of the prompt engine, wherein the detector is a machine learning-based detector, and wherein the detector comprises one or more of a keypoint detector, an object detector, a segmentation model, or any combination thereof; and
generating the input prompts with the prompt engine based on the one or more objects identified by the detector, wherein the input prompts comprise one or more of point prompts, box prompts, mask prompts, text prompts, or a combination thereof.

19. The non-transitory computer-readable medium of claim 16, wherein identifying the one or more segmentations of corrosion on the process equipment comprises:
identifying an unknown object in the raw image;
applying a zero-shot generalization model to the unknown object; and
fine tuning the pretrained generative AI model, wherein fine tuning the pretrained generative AI model comprises:
extracting one or more key components of the pretrained generative AI model, wherein the one or more key components comprises an image encoder, a prompt encoder, a mask decoder, or any combination thereof;
creating a custom image dataset for the pretrained generative AI model, wherein the custom image dataset comprises images of oil and gas process equipment;
setting up an optimizer and a scheduler for the pretrained generative AI model; and
iteratively updating the pretrained generative AI model with the custom image dataset.

20. The non-transitory computer-readable medium of claim 16, wherein:
identifying the one or more segmentations of corrosion on the process equipment does not comprise fine tuning the pretrained generative AI model;
determining the corrosion condition for each segmentation of the one or more segmentations of corrosion comprises determining, for each segmentation of the one or more segmentations of corrosion, a degree of corrosion, a corrosion pattern, a type of corrosion, or any combination thereof; and
displaying the annotated image comprises:
displaying a count of the segmentations of corrosion on the annotated image;
displaying the respective corrosion condition of each segmentation of the one or more segmentations of corrosion on the annotated image; and
displaying the one or more segmentations of corrosion as an overlay on the raw image.
